(19) United States
(12) Patent Application Publication
(10) Pub. No.: US 2023/0298693 A1
(43) Pub. Date: Sep. 21, 2023

(54) ALIGNMENT-FREE VARIANT CALLING

(71) Applicant: Rajant Health Incorporated, Malvern, PA (US)

(72) Inventors: Foad Nazari, Malvern, PA (US); Sneh Patel, Malvern, PA (US); Giana J. Schena, Malvern, PA (US); Emma K. Murray, Malvern, PA (US); Alina Sansevich, Malvern, PA (US)

(21) Appl. No.: 18/115,376

(22) Filed: Feb. 28, 2023

Related U.S. Application Data
(60) Provisional application No. 63/314,734, filed on Feb. 28, 2022.
(60) Provisional application No. 63/431,957, filed on Dec. 12, 2022.

(51) Int. Cl. G16B 20/20; G16B 20/10; G16B 40/20

(52) U.S. Cl. CPC G16B 20/20; G16B 20/10; G16B 40/20

(57) ABSTRACT

Disclosed is a methodology to find genetic sequence variations. In certain embodiments, a server receives a dataset comprised of a genetic sequence of control group RNA samples and experimental RNA samples and performs a count of unique k-mer sequences based on density values. Then, the server sorts the plurality of k-mer sequences based on their density values and applies a neighbor detection function to the plurality of k-mer sequences to identify one or more neighbor k-mer sequences to form one or more k-mer pair sequences. Then, the server filters the one or more k-mer pair sequences and merges the one or more filtered k-mer pair sequences into genetic variant candidates. The server then localizes the variant candidates in the reference genome to validate their existence and type and compares the plurality of genetic variant candidates against a variant database. The server then outputs the one or more identified sequence genetic variants.

### CROSS-REFERENCE TO RELATED APPLICATIONS

This application claims the benefit of U.S. Prov. App. No. 63/314,734, filed Feb. 28, 2022, and U.S. Prov. App. No. 63/431,957, filed Dec. 12, 2022, the entire contents of both of which are incorporated herein by reference.

### FIELD OF THE INVENTION

The present invention is directed to the field of next-generation sequencing, and more specifically, to its use in identifying genetic variants.

### BACKGROUND OF THE INVENTION

Detection of human genome variants is usually done using alignment-based approaches, which are based on mapping sequenced reads to the reference genome. Those approaches generally deliver highly accurate results when the sequences are closely related and can be aligned reliably. However, when the sequences are divergent, a reliable alignment cannot be performed. The alignment-based processes are also computationally complex and time-consuming, and so they are limited in application on large-scale sequence data.

With the recent advancements in Next Generation Sequencing (NGS), the amount of genomic data is being increased tremendously. That increase in the size of sequence data has caused challenges for alignment-based variant calling tools. Accordingly, there is a need for reliable, efficient variant calling methods to overcome the limitations of alignment-based approaches.

### SUMMARY OF THE INVENTION

The present invention comprises techniques for next-generation sequencing and its use in genetic analysis. In certain embodiments, a server receives a dataset comprised of a genetic sequence of control group RNA samples and experimental RNA samples and performs a count of unique k-mer sequences from the dataset. Then, the server sorts and filters the plurality of k-mer sequences based on density and applies a neighbor detection function to the plurality of k-mer sequences to identify one or more neighbor k-mer sequences to form one or more k-mer pair sequences. The neighbor detection function performs a k-to-3 dimensionality reduction transformation on the k-mers to reduce the computation cost. Then, the server filters the one or more k-mer pair sequences based on a predetermined edit distance and merges the one or more filtered k-mer pair sequences into a plurality of genetic variant candidates. The server then localizes the variant candidates in the reference genome to validate their existence and type, and also to check if there is any annotation associated with them in that specific location of the reference genome. The server subsequently compares the plurality of genetic variant candidates against a pre-populated variant database to specify if each detected one or more sequence genetic variants is novel or has been already annotated in the literature for the targeted disease and outputs the one or more identified sequence genetic variants through a graphic user interface.

In certain embodiments, the genetic variants identified are one or more of single nucleotide polymorphism (SNP), multiple nucleotide polymorphism (MNP), and insertion/deletion (INDEL).

In other embodiments, the dataset is comprised of FASTQ/A data for healthy individuals and unhealthy individuals.

In certain other embodiments, trimming of the genomic sequences is performed to remove unwanted or low-quality regions.

In certain other embodiments, sorting of the plurality of k-mer sequences is performed in descending order.

In certain embodiments, the sequence k-mers are filtered based on the ratio of k-mer density in one group vs the other.

In yet other embodiments, the server applies a T-test filter that performs an unequal variance T-test on the plurality of k-mer sequences.

In certain other embodiments, the k-mers are filtered based on the level that their density difference in control and experiment groups is compensated by its neighbor k-mers.

In certain embodiments, the k-mer pairs which have overlap are merged together to make longer sequence pairs.

In other embodiments, the output is in variant call format (VCF).

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a diagram of an exemplary embodiment of the hardware of the system of the present invention;

FIG. 2A is a flowchart showing the software processes of an exemplary embodiment of the present invention;

FIG. 2B is a flowchart showing the software processes of an exemplary embodiment of the present invention;

FIG. 3 is a graph showing a 3D representation of k-mers nucleotide counts; and

FIGS. 4A-4C show charts that demonstrate the geometry MD values (up to 6) with different colors in a 9*9 MD matrix.

### DETAILED DESCRIPTION OF THE PREFERRED EMBODIMENTS

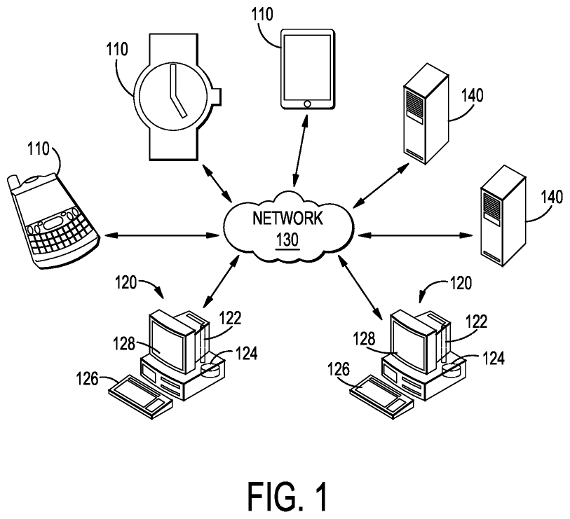

FIG. 1 is an exemplary embodiment of the health information system of the present invention. In the exemplary system 100, one or more peripheral devices 110 are connected to one or more computers 120 through a network 130. Examples of peripheral devices/locations 110 include smartphones, tablets, wearables devices, and any other electronic devices that collect and transmit data over a network that are known in the art. The network 130 may be a wide-area network, like the Internet, or a local area network, like an intranet. Because of the network 130, the physical location of the peripheral devices 110 and the computers 120 has no effect on the functionality of the hardware and software of the invention. Both implementations are described herein, and unless specified, it is contemplated that the peripheral devices 110 and the computers 120 may be in the same or in different physical locations. Communication between the hardware of the system may be accomplished in numerous known ways, for example using network connectivity components such as a modem or Ethernet adapter. The peripheral devices/locations 110 and the computers 120 will both include or be attached to communication equipment. Communications are contemplated as occurring through industry-standard protocols such as HTTP or HTTPS.

Each computer 120 is comprised of a central processing unit 122, a storage medium 124, a user-input device 126, and a display 128. Examples of computers that may be used are: commercially available personal computers, open source computing devices (e.g. Raspberry Pi), commercially available servers, and commercially available portable device (e.g. smartphones, smartwatches, tablets). In one embodiment, each of the peripheral devices 110 and each of the computers 120 of the system may have software related to the system installed on it. In such an embodiment, system data may be stored locally on the networked computers 120 or alternately, on one or more remote servers 140 that are accessible to any of the peripheral devices 110 or the networked computers 120 through a network 130. In alternative embodiments, the software runs as an application on the peripheral devices 110.

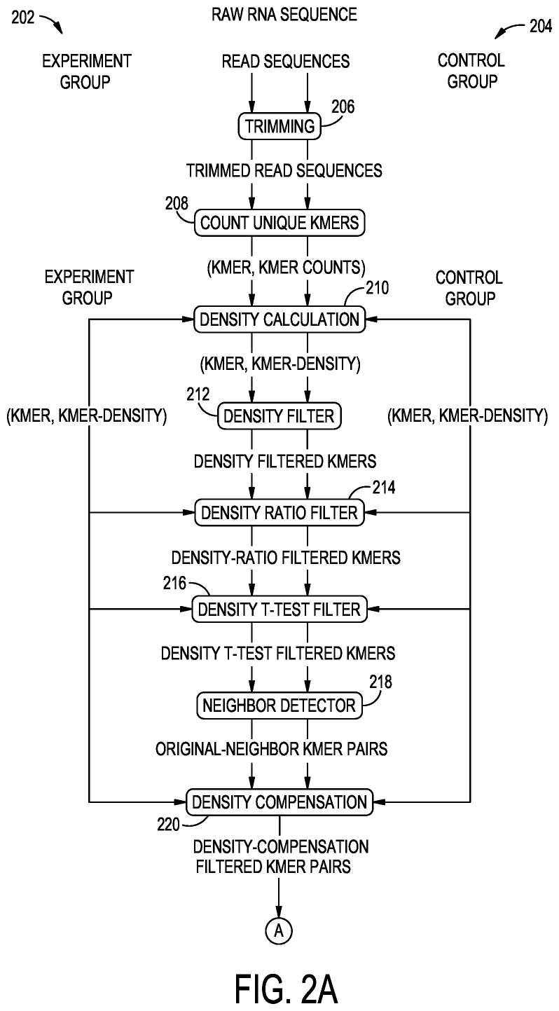

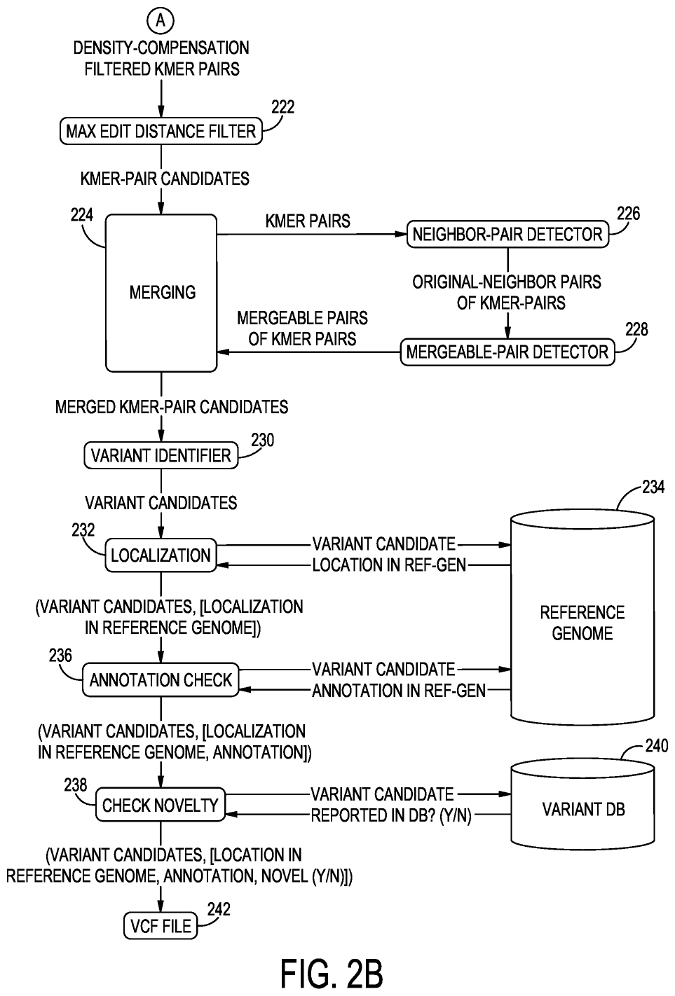

FIGS. 2A and 2B show a flow diagram of an alignment-free variant calling algorithm that may be used in accordance with the present invention to identify genomic variants using FASTQ/A data. Accurate variant calling in next-generation sequencing (NGS) data is a major step upon which virtually all downstream analysis and interpretation processes depend. The dataset that is used includes FASTQ/A data for individuals with healthy (control) and unhealthy (experimental) conditions, more specifically for one specific abnormality. Initially, the Control Group Raw Sequence Data 202 and the Experimental Group Raw Sequence Data 204 are transmitted to the Trimming module 206. At the Trimming module 206, the raw sequence data 202, 204 is trimmed, resulting in trimmed control group and experimental group raw sequence data. Sequence trimming is the process of removing unwanted or low-quality regions from a nucleotide or protein sequence. It can be done based on various criteria, such as quality scores, length, or the presence of contaminants or adapters. The goal of trimming is to improve the accuracy of downstream analysis, such as alignments and functional predictions. Trimming of adapter sequences from FASTQ/A reads is a common preprocessing step during NGS data analysis. Adapter removal is necessary to remove the adapter sequences from the 3′ end of the reads because those artificially added sequences (which are necessary to attach the DNA fragments to the flow cell and also barcoding) can interfere with the alignment of the reads to the genome.

The trimmed control group and experimental group raw sequence data are sent to the Count Unique k-mers module 208, at which the number of appearances of each unique k-mer in each sample is counted and recorded as its frequency. As a result, the output of this block for each one of control and experiment groups would be a {k-mer, [k-mer_count]} dictionary in which the key is the k-mer sequence, and the value is the [k-mer_count] which is frequency of the k-mer at each sample.

The {k-mer, [k-mer_count]} dictionary of the control group and the experimental group are then transmitted to the Density Calculation module 210. For each sample at the control and experimental groups, at the Density Calculation module 210, the density of k-mers is determined (for example, by determining the frequency of k-mers divided by total number of k-mers in that sample). Then, the mean of densities of each k-mer are calculated, for experimental and control groups, separately.

For each group, the k-mer densities and the mean of their densities are transmitted to the Density Filter module 212. At the Density Filter module 212, the k-mers in each group are sorted in descending order, based on their mean density values. In each group, the k-mers having a mean density beyond a predetermined density threshold pass this filter 212.

The k-mers from the control group that pass the Density Filter module 212 are transmitted to the Density Ratio Filter 214, which finds the corresponding mean density for each k-mer in the experiment group, calculates density_ratio^{control}(kmer), and in case the ratio is higher than a specific predetermined threshold, passes the k-mer through the filter 214. An exemplary calculation is shown below.

density_ratio^{control}(kmer)=mean_density^{control}(kmer)/mean_density^{experiment}(kmer) (1)

A similar filter is applied on the experiment group k-mers in which the k-mers from the experimental group that pass the Density Filter module 212 are transmitted to the Density Ratio Filter 214, which finds the corresponding mean density for each k-mer in the control group, calculates density_ratio^{experiment}(kmer), and in case the ratio is higher than a specific predetermined threshold, passes the k-mer through the filter 214. An exemplary calculation is shown below.

density_ratio^{experiment}(kmer)=mean_density^{experiment}(kmer)/mean_density^{control}(kmer) (2)

The Density T-Test module 216 is a filter which performs an unequal variance T-Test (Welch's T-test) on the k-mers that have passed the previous filter, using their densities and the density of their corresponding k-mers in the other group as well as the density ratios calculated by the Density Ratio Filter 214. That is an independent T-Test which is used when the number of samples in each group is different, and/or the variance of the two data sets is different. Preferably, the level of significance is assumed to be 5%. An exemplary calculation is shown below.

$${DoF}_{i} = ⌊ \frac{{( \frac{var ( {ρ}_{i}^{CNTL} )}{{Size}^{A}} + \frac{var ( {ρ}_{i}^{EXP} )}{{Size}^{B}} )}^{2}}{[ \frac{{( \frac{var ( {ρ}_{i}^{CNTL} )}{{Size}^{CNTL}} )}^{2}}{{Size}^{CNTL} - 1} + \frac{{( \frac{var ( {ρ}_{i}^{EXP} )}{{Size}^{EXP}} )}^{2}}{{Size}^{EXP} - 1} ]} ⌋ \qquad ( 3 )$$

In Equation 3, Size^{CNTL} and Size^{EXP} are the number of records (samples) in control and experiment groups, respectively. Also, var=σ²; in which var is variance and a is standard deviation, where the outer bracket in Equation 3 is the floor function.

Then the absolute value of the T-value is determined, as exemplarily calculated by Equation 4 below.

$$T {value}_{i} = \vert \frac{{ρ}_{i}^{CNTL} - {ρ}_{i}^{EXP}}{\sqrt{\frac{σ ( {ρ}_{i}^{CNTL} )}{{Size}^{CNTL}} + \frac{σ ( {ρ}_{i}^{EXP} )}{{Size}^{EXP}}}} \vert \qquad ( 4 )$$

Once the degrees of freedom (DOF) and significance level are known, a T-distribution critical value table gives the corresponding critical T-value. Now, if the calculated T-value for any k-mer is greater than or equal to the critical value, it passes the filter of the Density T-Test module 216. The Density T-Test module 216 is applied to the control and experiment group k-mers, separately.

For each filtered k-mer, the Neighbor Detector module 218 is used to find its neighbor k-mers (i.e., their hamming distance is less than a specific threshold) for further filtering downstream. Performing this process in k-dimensions is computationally expensive, because it needs to calculate the hamming distance of each filtered k-mer with all other k-mers. The Neighbor Detector module 218, for each k-mer sequence that has passed the previous filter, finds its nt_count neighbors. The two sequences are considered to be nt_count neighbors if the Manhattan distance between their nt_count vectors is equal to or smaller than a specific threshold. However, finding the neighbors in 4-dimensions is still computationally expensive. The Neighbor Detector module 218 transforms a 4D analysis into 3D, in which the neighbor sequences for a desired distance to the original k-mer form a predetermined (complex) geometry and are therefore easy to find.

TABLE 1
Example - Manhattan distance between nt-count vectors

| base | A | C | G | T |
|---|---|---|---|---|
| seq 1 | 5 | 4 | 3 | 2 |
| seq 2 | 4 | 5 | 3 | 2 |
| Difference (absolute value) | 1 | 1 | 0 | 0 |

| | |
|---|---|
| Manhattan Distance | 2 |

Further, where the nt_count vector is: [n_{A}, n_{C}, n_{G}, n_{T}]. since the number of nucleotides in each k-mer is k, we have:

n_{A}+n_{C}+n_{G}+n_{T}=k (5)

### And so,

n_{T}=k−(n_{A}+n_{C}+n_{G}) (6)

Therefore, a 3D_nt_count vector which is [n_{A}, n_{C}, n_{G}] can be used instead of nt_count since it has the same information for k-mers. With that, the dimension is reduced to 3.

The analysis then is subject to the following constraints:

n_{A}+n_{C}+n_{G}=<k (7)

0=<(n_{A},n_{C},n_{G})=<k (8)

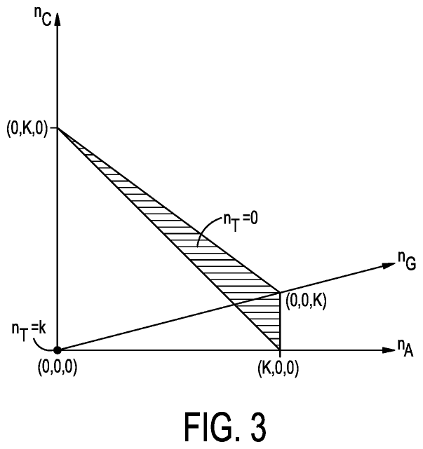

These two constraints make the possibility space a tetrahedron, as shown in FIG. 3, which shows a 3D representation of k-mers nucleotide counts. Therefore, there will be no k-mer outside this tetrahedron. The difference vector of a first and a second nt_count vectors can be presented as:

[n_{A}¹,n_{C}¹,n_{G}¹,n_{T}¹]−[n_{A}²,n_{C}²,n_{G}²,n_{T}²?]=[a,b,c,d] (9)

Therefore, we have:

a,+b+c+d=0 (10)

### And so:

d=−(a+b+c) (11)

The Manhattan distance between these two nt_count vector is:

MD=|a|+|b|+┌c┐+|d| (12)

With replacing d in this equation we will have:

MD=|a|+|b|+┌c┐+|a+b+c| (13)

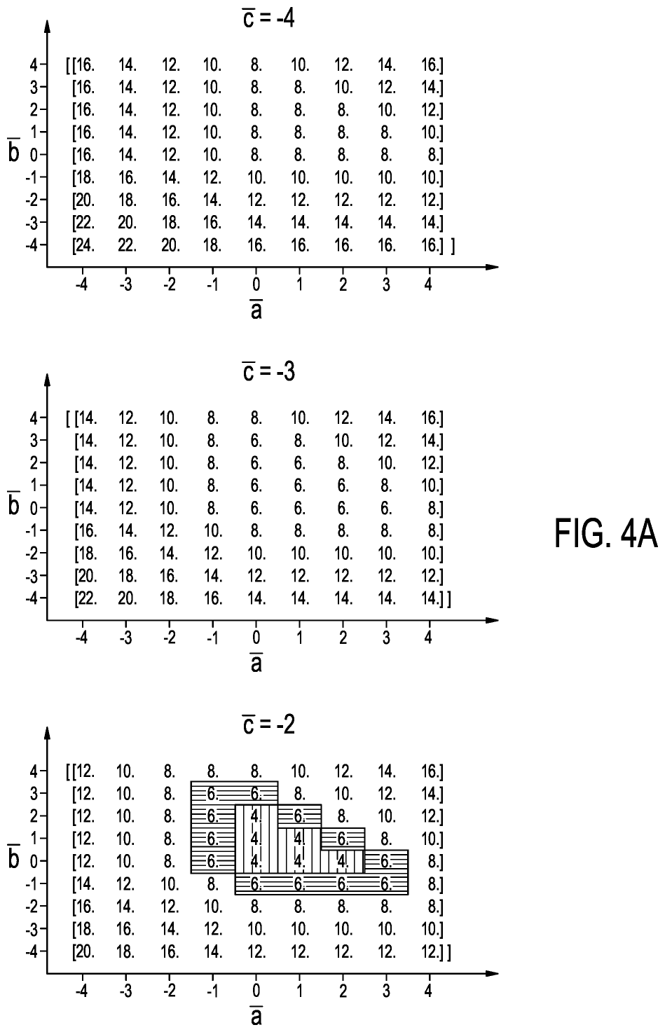

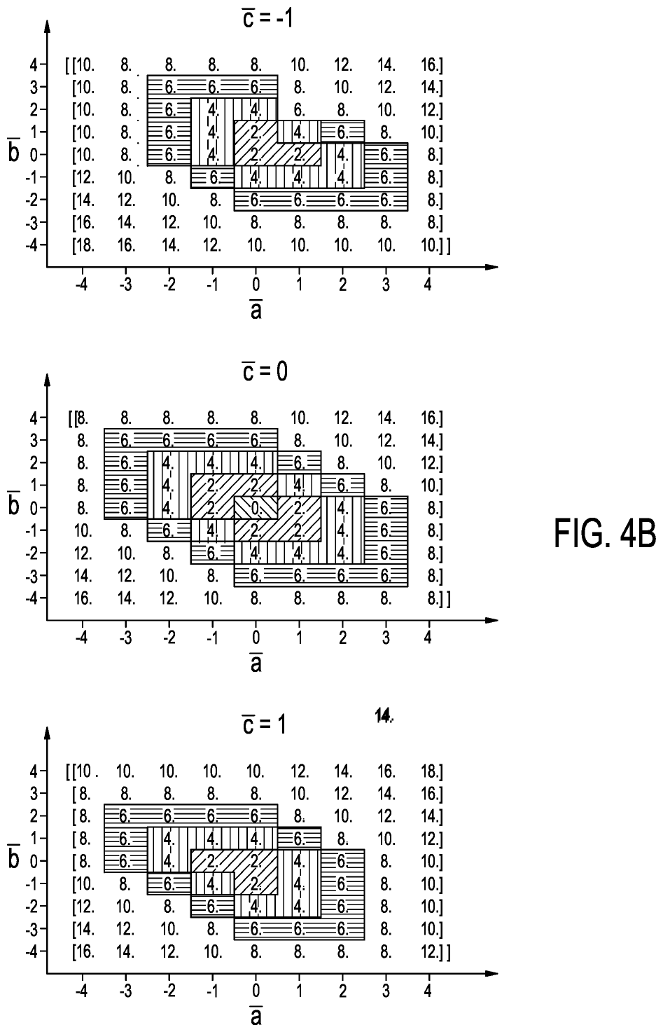

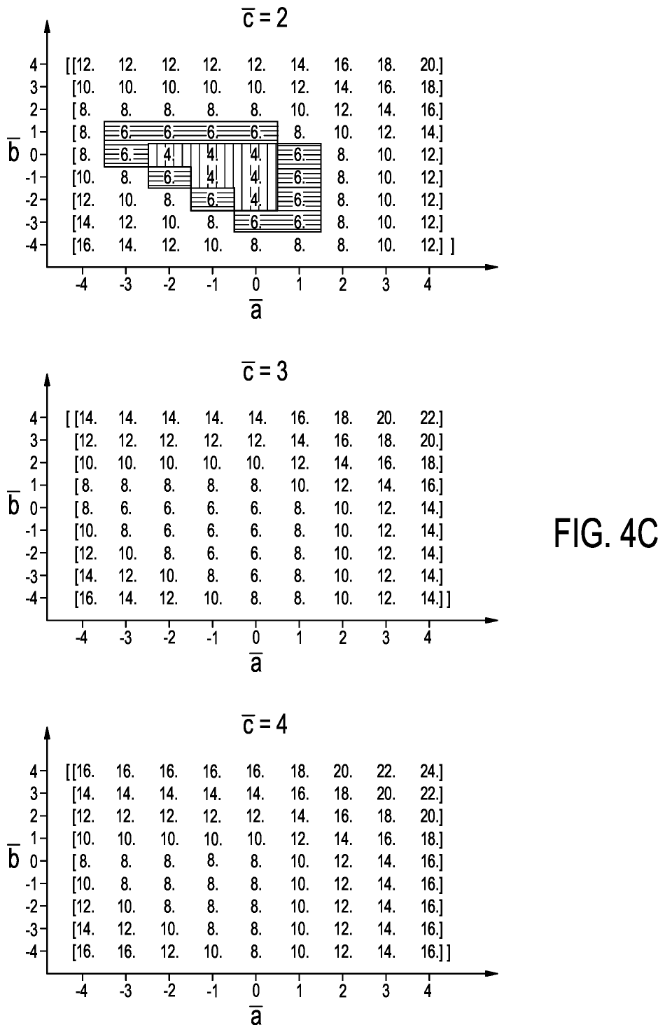

The 3D matrix of 3D_nt_count coordinates is called 3D_static. The way the Neighbor Detector module 218 works is that all experiment group k-mers locations in the 3D_static matrix are specified. We can find the position of any control group k-mer based on its 3D_nt_count coordinate. We found the geometry of the isodistance cells with every Manhattan distance of MD=md. For any desired md distance, we just need to go to isodistance cells that pick the experimental k-mers. They are experiment groups nt_count neighbors of the original control group k-mer with MD distance. FIGS. 4A-4C show the geometry MD values (up to 6) with different colors in a 9*9 MD_matrix. A similar process is performed by switching the groups, to find the control group nt_count neighbors of the original experiment group k-mers.

The geometry for isodistance cells for an MD_matrix is known and consistent across the 3D_static matrix and is subject to the tetrahedron surfaces constraints. It is proved that for any k value, the total number of cells in the 3D_static matrix is:

$${Cell\_number}_{3 D\_static} = \frac{( {( k + 1 )}^{3} + 5 ( k + 1 ) )}{6} \qquad ( 14 )$$

For MD=md, the total number of cells in MD_matrix is:

Cell_number_{MD}_{_{distance}}_{=md}=(15*md−18) (15)

For MD<=md, the total number of cells in MD_matrix is:

Cell_number_{MD}_{_{distance}}_{≤md}=(3.75*md²−1.5*md+1) (16)

For example, for k=18, the total number of cells in the 3D_static matrix is 1793 but the number of cells that have the MD=<2 (i.e., SNP) is 13. That means that with this neighbor detection method, the k-dimension hamming distance evaluation in Density Compensation Filter 220 for each control k-mer is just applied on the experiment k-mers located at 13 cells instead of 1793 possible cells, and vice versa

The k-mer variant candidates identified by the Neighbor Detector module 218 are transmitted to the k-mer Density Compensation Filter module 220, which further filters them.

For single nucleotide polymorphisms (SNPs), it is assumed that if a SNP occurs in a specific k-mer in a dataset, the density of that k-mer sequence is reduced and the density of the sequence with the changed nucleotide is increased. Table 2 shows an example for a dataset with a total 100 k-mers.

TABLE 2
SNP

| | | Control Dataset | | Experiment Dataset | |
|---|---|---|---|---|---|
| Name | k-mer | Frequency | Density | Frequency | Density |
| Original | ACTCCCTGCA | 7 | 0.07 | 3 | 0.03 |
| Neighbor | ACTCCCAGCA | 3 | 0.03 | 7 | 0.07 |

Based on that assumption, in an ideal scenario, the sum of the density of original and neighbor k-mers at each of the control and experiment datasets should be the same. Based on that idea, when there is an SNP, we expect that the absolute value of the average of Δρ of experiment and control groups would be smaller than the absolute value of Δρ of each experiment and control groups, separately, in which Δρ=(mean density^{control}−mean density^{experiment}). To do that, we define the ψ metric, which is related to the change in mean density of the original and neighbor k-mers due to each kind of variant, as follows.

$${ψ}_{i} = \vert \frac{Δ ( ( {ρ}_{i}^{o} + {ρ}_{i}^{n} / 2 )}{Δ ( {ρ}_{i}^{o} )} \vert \qquad ( 17 )$$

Insertion:

For single insertion, the process is similar to SNP, the only difference is the insertion neighbor has a nucleotide inserted in the insertion point of the original sequence and so has one nucleotide less from one end. Table 3 below shows an exemplary insertion and how it affects the dataset.

TABLE 3
Insertion

| | | Control Dataset | | Experiment Dataset | |
|---|---|---|---|---|---|
| Name | k-mer | Frequency | Density | Frequency | Density |
| Original | ACTCCCTGCA | 7 | 0.07 | 3 | 0.03 |
| Neighbor | ACTCCCATGC | 3 | 0.03 | 7 | 0.07 |

Deletion:

For single deletion, the process is similar to SNP, the only difference is the deletion neighbor has a nucleotide deleted from the deletion point of the original sequence, and so has one nucleotide more at one end.

The same process applies for multiple nucleotide polymorphism (MNP), multiple insertion or multiple deletion, only the number of varied nucleotides is greater than 1.

### Example: Larger Indels

seq_1:
ACTCCCTGCA (normal sequence)
seq_2.1:
ACTGGCCCTG (sequence with insertion)
seq_2.2:
ACTCTGCATT (sequence with deletion)

### Example: MNP

seq_1:
ACTCCCTGCA (normal sequence)
seq_2:
ACGGCCGCAA (sequence with MNP)

For the above, if ψ is smaller than a specific predetermined threshold, the pair (original & neighbor) k-mer sequences passes the Density Compensation Filter module 220.

The output original-neighbor k-mer pairs of the Density Compensation Filter module 220 are transmitted to the Max Edit Distance Filter 222, where the Needleman_Wunch approach is used to filter them further. The original-neighbor k-mer pairs whose edit distance is less than a specific threshold pass this filter. The edit distance between two sequences is a measure of the minimum number of operations (such as insertions, deletions, or substitutions) required to transform one sequence into the other.

The Needleman-Wunsch algorithm is a dynamic programming approach which is being used in bioinformatics to align two sequences. The algorithm creates a similarity matrix between the two sequences, considering gaps and mismatches, and then applies this matrix to find the optimal global alignment with the highest similarity score. The output of the Needleman-Wunsch algorithm is a pairwise sequence alignment with the highest possible similarity score, which reflects the evolutionary relationship between the two sequences.

In certain embodiments, the Density Compensation Filter module 220 may be applied after the Max Edit Distance Filter module 222, because the Max Edit Distance Filter module 222 is where original-neighbor k-mer pair potentially includes a variant is determined (i.e., the aforementioned SNP, MNP, Indel examples). However, since the Max Edit Distance Filter module 222 is computationally much more expensive than the Density Compensation Filter module 220, the latter module is typically applied first to reduce the load of the former (the number of pairs that go through the Density Compensation Filter module 220). Regardless of which filter is applied first, however, the same result is achieved.

The k-mer pair sequences that result from the Max Edit Distance Filter 222 are transmitted to the Merging module 224. Many of the k-mer-pair sequences that have been identified and sent to the Merging module 224 may have overlap. For example, three k-mer pairs that are presented in Table 4 below can be merged together to create a bigger sequence, so that instead of sending three pairs of sequences to the downstream modules of the software, just one bigger sequence will be sent which will result in less computation. It will be computationally expensive to check each two pairs together if they are mergeable. So, there is a need to reduce the number of candidates for merging to each pair and then evaluate them, which is performed at the Merging module 224.

TABLE 4
Merging Example

| | K-mer Sequences | |
|---|---|---|
| | Control | Experiment |
| Pair 1 | AAAAAAAAAAAAGAA | AAAAAAAAAAACAGAA |
| Pair 2 | TAAAAAAAAAAAAAGA | TAAAAAAAAAAACAGA |
| Pair 3 | TTAAAAAAAAAAAAAG | TTAAAAAAAAAAACAG |
| Merged | TTAAAAAAAAAAAAAGAA | TTAAAAAAAAAAACAGAA |
| Pair | | |

All the pairs are sent from the Merging module 224 to the Neighbor Pair Detector 226 and then to the Mergeable Pair Detector module 228 to find the merable pairs for each k-mer-pair. Those pairs which have a right side mergeable pair (as explained with regard to the Mergeable Pair Detector module 228) are merged, and those which do not, will remain not-merged.

The Neighbor Pair Detector 226 does a similar job to the Neighbor Detector module 218, the only difference being that for a k-mer pair to be a neighbor pair of a given pair, its experiment and control nt-vectors should be at the neighborhood of the nt-vectors of the experiment and control sequences of the given pair, respectively. So, to find which two pairs are mergeable together, we first create the nt-vector for each k-mer of each candidate pair. For any given pair, called an original-pair, any other pair that the Manhattan distance between the nt-vector of their control k-mers and their experiment k-mers are less than a specific threshold (here, threshold=2), (i.e., both the Manhattan distance between the control k-mers and the Manhattan distance between the experiment k-mers were smaller than a threshold), is considered as the nt-count neighbor-pair of the original pair. The example shown in Table 5 below illustrates that assuming the Manhattan distance threshold of 2, (pair1, pair2), (pair2, pair1), (pair2, pair3) and (pair3, pair2) are original-neighbor pairs.

TABLE 5
Neighbor Pair Detections

| | | | | | nt-vector Manhattan | | |
|---|---|---|---|---|---|---|---|
| | | | Control | | distance with | | |
| | | | nt-vector | | other pairs | | |
| | K-mer Sequences | | [A,C,G,T] | | (Control, Experiment) | | |
| | Control | Experiment | Control | Experiment | Pair 1 | Pair 2 | Pair 3 |
| Pair 1 | AAAAAAAAA | AAAAAAAAAA | [15,0,1,0] | [14,1,1,0] | | (2,2) | (4,4) |
| | AAAAGAA | ACAGAA | | | | | |
| Pair 2 | TAAAAAAAA | TAAAAAAAAA | [14,0,1,1] | [13,1,1,1] | (2,2) | | (2,2) |
| | AAAAAGA | AACAGA | | | | | |
| Pair 3 | TTAAAAAAA | TTAAAAAAAA | [13,0,1,2] | [12,1,1,2] | (4,4) | (2,2) | |
| | AAAAAAG | AAACAG | | | | | |
| Merged | TTAAAAAAA | TTAAAAAAAA | | | | | |
| Pair | AAAAAAGAA | AAACAGAA | | | | | |

For all detected pairs, if the first k−1 nucleotides of the original-pair were the same as the last k−1 of the neighbor-pair, the latter is labeled as the right side mergeable pair (RSMP) of the former, and vice versa. That process is done for all pairs, as exemplarily shown in Table 6 below.

TABLE 6
Mergeable Pairs

| | | | Control nt-vector | | |
|---|---|---|---|---|---|
| | K-mer Sequences | | [A,C,G,T] | | |
| | Control | Experiment | Control | Experiment | Label |
| Pair 1 | AAAAAAAAA | AAAAAAAAAA | [15,0,1,0] | [14,1,1,0] | RSMP of |
| | AAAAGAA | ACAGAA | | | Pair 2 |
| Pair 2 | TAAAAAAAA | TAAAAAAAAA | [14,0,1,1] | [13,1,1,1] | RSMP of |
| | AAAAAGA | AACAGA | | | Pair 3 |
| Pair 3 | TTAAAAAAA | TTAAAAAAAA | [13,0,1,2] | [12,1,1,2] | |
| | AAAAAAG | AAACAG | | | |
| Merged | TTAAAAAAA | TTAAAAAAAA | | | |
| Pair | AAAAAAGAA | AAACAGAA | | | |

Then, both merged and not-merged pairs are transmitted from the Merging module 224 to the Variant Identifier module 230. At the Variant Identifier module 230, the Needleman-Wunch matrix of the experiment vs control sequence of each candidate pair is created. Based on that matrix, the location and type of variation is specified.

The result of the Variant Identifier module 230 is then validated at the Localization module 232, which validates and confirms the detected variants, exemplarily by localizing the variants in the Reference Genome module 234, using existing tools. Then, at the Annotation Check module 236, the variants are checked to confirm if there is any annotation associated with the detected variant in that specific location of the reference genome. This analysis is again performed by comparison to the data found in the Reference Genome module 236. The detected variants and their annotation are then transferred to the Check Novelty module 238.

The Check Novelty module 238 functions to check if the detected variants are already annotated for the targeted disease in its databases. A list of variants which are already identified and found in the system databases to have a causation or correlation relationship with the abnormality being analyzed are collected at the Variant DB module 240, in advance. The Check Novelty module 238 compares the detected merged variant candidates against that variant list of Variant DB 240 to see which variant is already known to be associated with the targeted disease or abnormality and which is novel and does not appear in the database. Based on that determination, each detected variant can be designated as either “new” or “existing” in candidate variants.

Following the Check Novelty module 238, the result of the variant caller processes described is presented in the standard VCF format by the VCF Output module 242.

The foregoing description and drawings should be considered as illustrative only of the principles of the invention. The invention is not intended to be limited by the preferred embodiment and may be implemented in a variety of ways that will be clear to one of ordinary skill in the art. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention.

1. A system for variant analysis comprised of a server that:
receives a dataset comprising one or more control genetic sequence samples and one or more experimental genetic sequence samples;
performs a count of unique k-mer sequences from the dataset;
sorts the plurality of k-mer sequences based on density;
applies a neighbor detection function to the plurality of k-mer sequences to identify one or more neighbor k-mer sequences to form one or more k-mer pair sequences;
filters the one or more k-mer pair sequences based on a predetermined edit distance;
merges the one or more filtered k-mer pair sequences into a plurality of genetic variant candidates;
compares the plurality of genetic variant candidates against a pre-populated variant database to specify if each detected sequence genetic variant is novel or has been already annotated in a targeted disease; and
outputs the one or more identified sequence genetic variants through a graphic user interface.

2. The system of claim 1, wherein the neighbor detection function comprises a dimensionality reduction transformation on the plurality of k-mer sequences.

3. The system of claim 1, wherein the genetic variants are one or more of single nucleotide polymorphism (SNP), multiple nucleotide polymorphism (MNP), and insertion/deletion (INDEL).

4. The system of claim 1, wherein the dataset is comprised of RNA data in a FASTQ/A format for healthy individuals and unhealthy individuals.

5. The system of claim 1, wherein the server further trims low quality regions from the control genetic sequence samples and the experimental genetic sequence samples of the dataset.

6. The system of claim 1, wherein the sorting of the plurality of k-mer sequences is performed based on their density values in descending order.

7. The system of claim 1, wherein the server further filters the plurality of k-mer sequences by calculating a ratio of k-mer density in one subset of the plurality of k-mer sequences as compared to a second subset of the plurality of k-mer sequences.

8. The system of claim 1, wherein the server further applies a T-test filter that performs an unequal variance T-test on the plurality of k-mer sequences.

9. The system of claim 1, wherein the filtering of the one or more k-mer pair sequences is based on the amount of density difference in the control genetic sequence samples and experimental genetic sequence samples as compensated by their neighbor k-mer sequences.

10. The system of claim 1, wherein the one or more filtered k-mer pair sequences are merged based on overlap.

11. The system of claim 1, wherein server further localizes the plurality of genetic variant candidates in a reference genome to validate their existence and type.

12. The system of claim 1, wherein the server further performs a check on the plurality of genetic variant candidates to determine whether an annotation is associated with said genetic variant candidates at a specific location on the reference genome.

13. The system of claim 1, wherein the output is in variant call format (VCF).

14. A computer-implemented method for variant analysis comprising:
receiving a dataset comprising one or more control genetic sequence samples and one or more experimental genetic sequence samples;
performing a count of unique k-mer sequences from the dataset;
sorting the plurality of k-mer sequences based on density;
applying a neighbor detection function to the plurality of k-mer sequences to identify one or more neighbor k-mer sequences to form one or more k-mer pair sequences;
filtering the one or more k-mer pair sequences based on a predetermined edit distance;
merging the one or more filtered k-mer pair sequences into a plurality of genetic variant candidates;
comparing the plurality of genetic variant candidates against a pre-populated variant database to specify if each detected sequence genetic variant is novel or has been already annotated in a targeted disease; and
outputting the one or more identified sequence genetic variants through a graphic user interface.

15. The method of claim 14, wherein the neighbor detection function comprises a dimensionality reduction transformation on the plurality of k-mer sequences.

16. The method of claim 14, wherein the genetic variants are one or more of single nucleotide polymorphism (SNP), multiple nucleotide polymorphism (MNP), and insertion/deletion (INDEL).

17. The method of claim 14, wherein the dataset is comprised of RNA data in a FASTQ/A format for healthy individuals and unhealthy individuals.

18. The method of claim 14, further comprising trimming low quality regions from the control genetic sequence samples and the experimental genetic sequence samples of the dataset.

19. The method of claim 14, wherein the sorting of the plurality of k-mer sequences is performed based on their density values in descending order.

20. The method of claim 14, further comprising filtering the plurality of k-mer sequences by calculating a ratio of k-mer density in one subset of the plurality of k-mer sequences as compared to a second subset of the plurality of k-mer sequences.

21. The method of claim 14, further comprising applying a T-test filter that performs an unequal variance T-test on the plurality of k-mer sequences.

22. The method of claim 14, wherein the filtering of the one or more k-mer pair sequences is based on the amount of density difference in the control genetic sequence samples and experimental genetic sequence samples as compensated by their neighbor k-mer sequences.

23. The method of claim 14, wherein the one or more filtered k-mer pair sequences are merged based on overlap.

24. The method of claim 14, wherein server further localizes the plurality of genetic variant candidates in a reference genome to validate their existence and type.

25. The method of claim 14, further comprising performing a check on the plurality of genetic variant candidates to determine whether an annotation is associated with said genetic variant candidates at a specific location on the reference genome.

26. The method of claim 14, wherein the output is in variant call format (VCF).
